(19) United States
(12) Patent Application Publication
(10) Pub. No.: US 2011/0276114 A1
(43) Pub. Date: Nov. 10, 2011

(54) Body Cooling Apparatus

(76) Inventor: Husain S.A. Faridoon, Dasman (KW)

(21) Appl. No.: 12/773,139

(22) Filed: May 4, 2010

(51) Int. Cl. A61F 7/00

(52) U.S. Cl. 607/104

(57) ABSTRACT

A body cooling apparatus for cooling a human being includes a cooling chamber with a copper plate adjacent to or in contact with the cooling chamber. The copper plate has two sides one of which is exposed to cooled refrigerant while the other side is exposed to a mass of water that is circulated through a pump through a tubular member to reach various parts of the human body to thereby cool the various parts of the body.

### FIELD OF THE INVENTION

This invention relates to a body cooling apparatus and more particularly to a body cooling apparatus that cools various parts of a human body that are exposed to a high ambient temperature.

### BACKGROUND FOR THE INVENTION

Apparatus and clothing for cooling individuals in excessively warm climates are well known and been in use for many years. For example, a Cool Coat is disclosed in a U.S. Patent of Troyer, No. 3,610,323. As disclosed therein, a thermal coat or garment for maintaining a person comfortably cool includes an article of clothing having a tube or tubular network that makes up the passage way for circulating cooling water and other chemicals such as Freon refrigerant. The tubes house capillary openings through the wall thereof to permit the tube to evaporate outwardly to create a cooling effect to the wearer.

A more recent approach to body cooling apparatus is disclosed in a U.S. Pat. No. 4,998,415 of Larsen. As disclosed therein, the body cooling apparatus removes body or external heat in an environment where sufficient cooling by perspiration is not available. The apparatus includes a compressor and a condenser which feeds liquid coolant to a flexible tube network which is held adjacent the body to be cooled. The cooling takes place by the boiling of the liquid within the flexible tube network. The flow of liquid into the network is controlled depending upon the amount of cooling needed.

Finally, a Miniaturized Thermoelectric Apparatus for Air Conditioning a Protective Body Suit is disclosed in a U.S. Pat. No. 5,197,294 of Galvan et al. As disclosed, the apparatus comprises an assembly made up of a Peltier effect thermoelectric device in the form of bimetallic or plurimetalic plates connected to a low voltage D.C. power supply. An opposed cold and hot surface are in contact with respective heat exchangers. The assembly is contained in a housing in which two distinct and separate conduits are provided for the forced flow of air through the respective ones of said heat exchangers.

Notwithstanding the above, it is presently believed that there is a need and a potential commercial market for an improved body cooling apparatus in accordance with the present invention. There should be a market for such apparatus because it is light weight, relatively small and of simplified design and construction. It is also believed that such apparatus is rugged and can be produced and sold at a competitive price.

### BRIEF SUMMARY OF THE INVENTION

In essence a body cooling apparatus for cooling a human body that is exposed to an ambient high temperature includes a cooling chamber. The cooling chamber includes a copper plate having a first side and a second side and refrigeration means together with means for exposing the first side of the copper plate to the cooling effect of the refrigeration means. A mass of water and means for bringing the mass of water into contact with the second side of the copper plate when the first side of the copper plate is cooled by the refrigeration is also provided. In addition, the apparatus includes a tubular member for cooling a portion of a human body and means for circulating the mass of cooled water through the tubular member to thereby cool the human body.

A preferred embodiment of the invention includes a cooling chamber including a copper plate having first and second sides and wherein the cooling chamber is divided into two separate chambers. The apparatus also includes refrigeration means including a compressor, a condenser, an expansion valve and evaporator and a mass of refrigerant and wherein the evaporator is adjacent to or in contact with the first side of the copper plate. The second embodiment of the invention also includes a mass of water and a pump for bringing the mass of water into the second portion of the chamber and into contact with the second side of the copper plate while the first side of the copper plate is cooled by the refrigeration means. Further, a tubular member extends to portions of a human body for cooling the human body and the pump circulates cooling water through the tubular member to thereby cool the water and subsequently the human body.

The invention will now be described in connection with the accompanying drawings wherein like reference numerals have been used to indicate like parts.

### DESCRIPTION OF THE DRAWINGS

FIG. 1 is a schematic illustration of a human figure with a device in accordance with the present invention applied to the arms, legs and body of an individual;

FIG. 2 is a schematic illustration of the refrigeration unit and water circulating means of a vapor compression refrigeration system, water circulating system and cooling chamber as used in the present invention; and

FIG. 3 is a schematic illustration of a Peltier electric cooling device for use in a second embodiment of the invention.

### DESCRIPTION OF THE PREFERRED EMBODIMENTS OF THE INVENTION

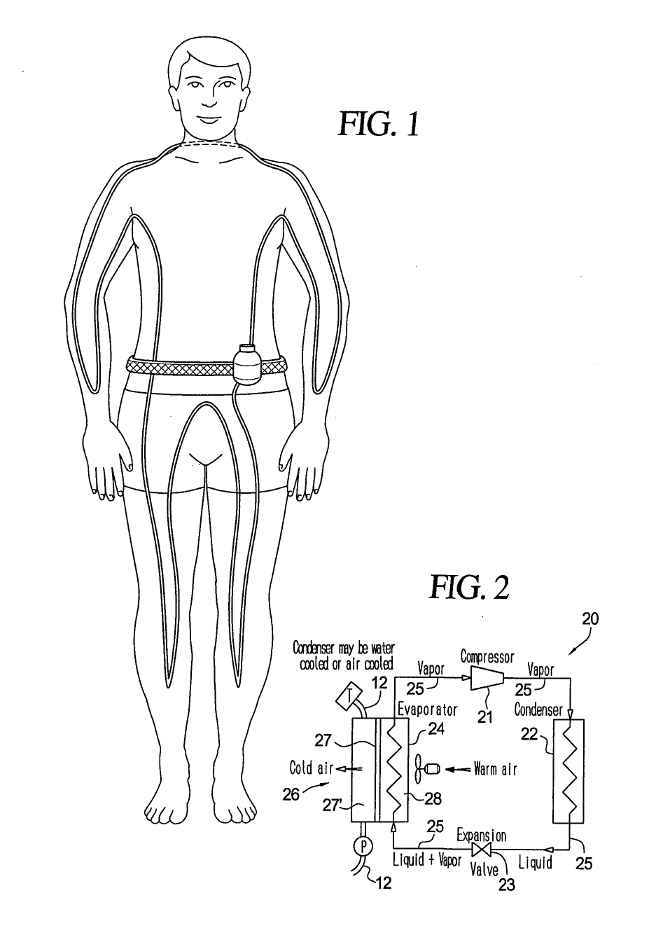

As illustrated in FIG. 1 an outline of a human body with a body cooling apparatus 10 includes a tubular member or cooling tube 12 extending upwardly out of a refrigeration/cooling circulator 14 that extends across the chest of an individual and down and back of an arm and across the back of the neck of the individual. The cooling tube extends down and back along the second arm, down across the chest and abdomen and down and back a leg of the individual. It then extends across the abdomen, down and back the second leg and upward to the waist of the individual and return to the refrigeration/cooling circulating unit 14.

It should be recognized that the cooling tube could be wound around the upper body as well as an arm and leg for more complete cooling of an individual's body, but would require a large capacity pump for moving more coolant such as water and a larger capacity refrigeration unit.

Refrigeration means such as a vapor compressor refrigeration unit are shown in FIG. 2. The refrigeration unit 20 includes a compressor 21, condenser 22, expansion valve 23, and evaporator 24 connected together by a conduit or piping 25. As illustrated the refrigeration unit 20 includes a cooling chamber 26 with a copper plate 27 that separates the cooling chamber into two segments or sub-chambers 27′ and 28. The refrigeration unit contains a mass of conventional refrigerant such as halo alkane, namely HCFC (R-22 or an HFC (R-134) or other appropriate refrigerant as will be well understood by persons of ordinary skill in the art.

For example, as described in more detail in Wikipedia a vapor compression refrigeration system uses a circulating liquid refrigerant as the medium which absorbs and removes heat from a space or object to be cooled and subsequently rejects the heat elsewhere. A typical system, a single-state vapor compression system has four components, a compressor, a condenser, an expansion valve and an evaporator. Circulating refrigerant enters the compressor in the thermodynamic state known as saturated vapor and is compressed to a higher pressure, resulting in a higher temperature. The hot, compressed vapor is then in the thermo dynamic state known as a super heated vapor and it is at a temperature and pressure at which it can be condensed with typically available cooling water or cooling air. The hot vapor is routed to a condenser where it is cooled and condensed into a liquid by flowing through a coil or length of tube with cool water or cool air flowing across the coil or tubes. This is where the circulating refrigerant rejects heat from the system and the rejected heat is carried away by either the water or the air.

The condensed liquid refrigerant is in the thermodynamic state known as saturated liquid and is next routed through an expansion valve where it undergoes an abrupt reduction in pressure. That pressure reduction results in the adiabatic flash evaporation of a part of the liquid refrigerant. The auto-refrigeration effect of the adiabatic flash evaporation lowers the temperature of the liquid and vapor refrigerant mixture to where it is colder than the temperature of the enclosed space or in this case the copper plate to be refrigerated.

The cold mixture is then routed through the coil or tubes to the evaporator. A fan circulates the warm air in the enclosed space across the coil or tubes carrying the cold refrigerant liquid and vapor mixture. The warm air evaporates the liquid part of the cold refrigerant mixture. At the same time, the circulating air is cooled and thus lowers the temperature of an enclosed space to the desired temperature. The evaporator is where the circulating refrigerant absorbs and removes heat which is subsequently rejected in the condenser and transferred elsewhere by the water or air used in the condenser.

To complete the refrigeration cycle, the refrigerant vapor from the evaporator is again a saturated vapor and is routed back into the compressor.

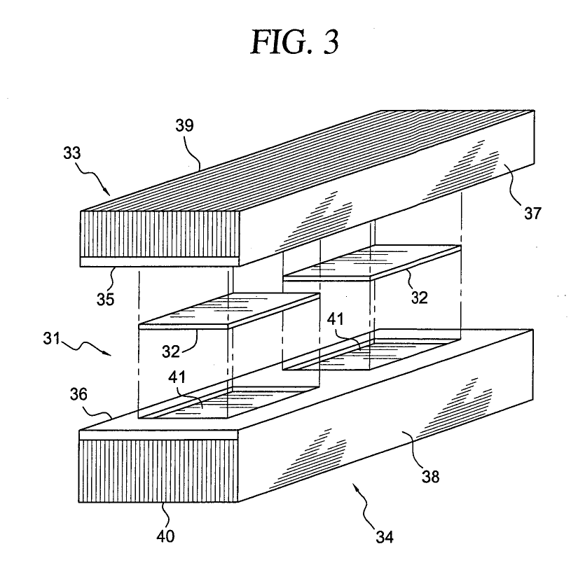

As illustrated in FIG. 3 a heat exchange unit 31 of the type used in the second embodiment of the invention includes two Peltier effect plates 32 made of a quaternary alloy Bi—Te—Se—Sv with suitable doping, treated to obtain a polycrystalline orientated state as will be well understood by persons of ordinary skill in the art of Peltier effect heating/cooling units

Although not shown, the plates 32 are conventionally connected to a source of low voltage DC power supply as a function of the polarity to obtain a transfer of heat from one surface to the opposite surface of the plates 32.

In this embodiment, it is assumed that the polarity of the electric power supply is such that the upper surface of plate 2 forms the cold surface while the lower surface forms the hot surface. A first and a second heat exchanger 33 and 34 respectively are formed by respective base walls 35 and 36 of a generally rectangular shape each one integral with respective perpendicular edges 37 and 38 on the long sides between which are placed a plurality of fins 39 and 40 fastened at their lower ends to the internal surface of the base walls 34 and 35 in a closely spaced relationship parallel to each other and to the respective edges 37 and 38.

The material forming the heat exchangers 33 and 34 is a thermally conductive metal as for example aluminum.

In the external surfaces of the base walls 35 and 36 of each exchanger 33 and 34 are formed housings for the plates 32, the housings are shown only in relation to the heat exchanger 34 and being indicated with the numeral 41 having a depth equal to about half the thickness of the plates 32 in such

While the invention has been described in connection with its preferred embodiments, it should be recognized that changes and modifications may be made therein without departing from the scope of the appended claims.

What is claimed is:

1. A body cooling apparatus for cooling a human body that is exposed to an ambient high temperature, said apparatus comprising:
a cooling chamber including a copper plate having a first side and a second side;
refrigeration means and means for exposing said first side of said copper plate to the cooling effect of said refrigeration means;
a mass of water and means for bringing said mass of water into contact with said second side of said copper plate when said first side of said copper plate is cooled by said refrigeration means; and
a tubular member for cooling a portion of a human body and means for circulating said mass of cooled water through said tubular member to thereby cool the human body.

2. A body cooling apparatus for cooling a human body that is exposed to an ambient high temperature according to claim 1 in which said refrigeration means is a vapor-compression refrigeration system.

3. A body cooling apparatus for cooling a human body that is exposed to an ambient high temperature according to claim 2 in which said refrigeration system includes a compressor, a condenser, an expansion valve and an evaporator and wherein said evaporator is adjacent to or in contact with said first side of copper plate.

4. A body cooling apparatus for cooling a human body that is exposed to an ambient high temperature according to claim 3 in which said refrigerant is a haloalkane refrigerant.

5. A body cooling apparatus for cooling a human body that is exposed to an ambient high temperature according to claim 3 in which said refrigerant is an HCFC (R-22) or an HFC (R-134a or E-410A).

6. A body cooling apparatus for cooling a human body that is exposed to an ambient high temperature according to claim 1 in which said refrigeration means is a Peltier effect cooler.

7. A body cooling apparatus for cooling a human body that is exposed to an ambient high temperature, said apparatus comprising:
a cooling chamber including a copper plate having a first side and a second side;
refrigeration means including a compressor, a condenser, an expansion valve, an evaporator and a mass of refrigerant, and wherein said evaporator is adjacent to or in contact with said first side of said copper plate;
a mass of water and a pump for bringing said mass of water into contact with said second side of said copper plate while said first side of said copper plate is cooled by said refrigeration means; and
a tubular member extending to portions of a human body for cooling the portions of the human body and said pump circulating cooled water through said tubular member to thereby cool the human body.

8. A body cooling apparatus for cooling a human body that is exposed to an ambient high temperature according to claim 7 that includes a temperature sensor for sensing the temperature of said cooled water.

9. A body cooling apparatus for cooling a human body that is exposed to an ambient high temperature according to claim 8 which includes a rechargeable battery for powering said apparatus.

10. A body cooling apparatus for cooling a human body that is exposed to an ambient high temperature according to claim 9 which includes a solar cell for recharging said rechargeable battery.

11. A body cooling apparatus for cooling a human body that is exposed to an ambient high temperature consisting of:
a cooling chamber including a copper plate having a first side and a second side;
refrigeration means including a compressor, a condenser, an expansion valve, an evaporator and a mass of haloalkane refrigerant and wherein said evaporator is adjacent to or in contact with said first side of said copper plate;
a mass of water and circulating means including a pump for bringing said mass of water into contact with said second side of said copper plate while said first side of said copper plate is cooled by said refrigeration means;
a tubular member extending from and connected to various parts of a human body from said cooling chamber and means including said circulating means and said pump for circulating said mass of water through said tubular member and against said second side of said copper plate to thereby circulate cooled water to various parts of the human body; and
a temperature sensor for sensing the temperature of said cooled water, a timer and a rechargeable batter for powering said apparatus and a solar panel for recharging said battery.
